(19) United States
(12) Patent Application Publication
(10) Pub. No.: US 2024/0343496 A1
(43) Pub. Date: Oct. 17, 2024

(54) CONTROL SYSTEM, CONTROL METHOD, AND NON-TRANSITORY STORAGE MEDIUM

(71) Applicant: TOYOTA JIDOSHA KABUSHIKI KAISHA, Toyota-shi (JP)

(72) Inventors: Daiki FUKUNAGA, Toyota-shi (JP); Shiro ODA, Anjo-shi (JP); Takeshi MATSUI, Nissin-shi (JP)

(73) Assignee: TOYOTA JIDOSHA KABUSHIKI KAISHA, Toyota-shi (JP)

(21) Appl. No.: 18/588,934

(22) Filed: Feb. 27, 2024

(30) Foreign Application Priority Data
Apr. 17, 2023 (JP) 2023-066887

(51) Int. Cl. B65G 35/00; G05D 1/656; G05D 105/28

(52) U.S. Cl. CPC B65G 35/00; G05D 1/656; B65G 2203/0216; B65G 2203/041; G05D 2105/28

(57) ABSTRACT

A control system controls a conveyance system including a transfer robot capable of autonomously moving and capable of conveying an article to be conveyed. The control system acquires conveyance destination information from a wagon capable of accommodating the article. The conveyance destination information indicates a destination of conveyance of the article. The control system causes the transfer robot to autonomously move such that the transfer robot conveys the wagon to the destination of conveyance indicated by the acquired conveyance destination information.

### CROSS-REFERENCE TO RELATED APPLICATION

This application claims priority to Japanese Patent Application No. 2023-066887 filed on Apr. 17, 2023, incorporated herein by reference in its entirety.

### BACKGROUND

### 1. Technical Field

The disclosure relates to a control system, a control method, and a non-transitory storage medium.

### 2. Description of Related Art

U.S. Pat. No. 9,020,679 describes a technology for managing assets in an asset management database by using a radio frequency identification (RFID) tag attached to each of the assets. With the technology described in U.S. Pat. No. 9,020,679, the location of an RFID tag attached to an asset is chosen based on scan data of the RFID tag, read by an autonomous mobile robot, the location is associated with a location on a map, and the location and direction of the autonomous mobile robot in the asset management database are updated.

### SUMMARY

A technology that a user easily issues conveying instructions with a designated destination of conveyance to a transfer robot is desired. The technology described in U.S. Pat. No. 9,020,679 cannot solve this task.

The disclosure provides a control system, a control method, and a non-transitory storage medium that allow a user to easily issue conveying instructions with a designated destination of conveyance to a transfer robot.

A first aspect of the disclosure provides a control system. The control system controls a conveyance system including a transfer robot capable of autonomously moving and capable of conveying an article to be conveyed. The control system includes: an acquisition unit configured to acquire conveyance destination information from a wagon capable of accommodating the article, the conveyance destination information indicating a destination of conveyance of the article; and a controller configured to cause the transfer robot to autonomously move such that the transfer robot conveys the wagon to the destination of conveyance indicated by the conveyance destination information. With the thus configured control system, when a user just causes the wagon to hold conveyance destination information in order to issue conveying instructions with a destination of conveyance of the article to the transfer robot, it is possible to acquire the conveyance destination information from the wagon and cause the transfer robot to autonomously move and convey the article to the destination of conveyance in accordance with the conveying instructions indicated by the conveyance destination information. Thus, with the control system, the user is able to easily issue conveying instructions with a designated destination of conveyance to the transfer robot. The autonomous movement may also be performed by using a learning model obtained through machine learning.

The wagon may include a label on which the conveyance destination information is printed, and the acquisition unit may include a first sensor configured to detect the conveyance destination information from the label or a communication unit configured to receive the conveyance destination information detected by a second sensor. With the thus configured control system, the user is able to easily issue conveying instructions without a device that needs electricity, and the transfer robot can receive the conveying instructions.

The wagon may include a label on which the conveyance destination information is printed, the acquisition unit may include a first camera configured to obtain image data by capturing an image on the label or a communication unit configured to receive image data obtained by capturing an image on the label with a second camera, and the controller may be configured to detect the conveyance destination information from the image data. With the thus configured control system, the user is able to easily issue conveying instructions without a device that needs electricity, and the transfer robot can receive the conveying instructions.

The conveyance destination information may be printed on a first side of the label, and moving destination information indicating a destination of movement to which the wagon is moved after being conveyed to the destination of conveyance indicated by the conveyance destination information may be printed on a second side of the label Thus, the user is able to easily issue instructions to return the wagon.

A two-dimensional code or a one-dimensional code, indicating the destination of conveyance, may be printed on the label. Thus, it is possible to reduce wrong recognition of the conveyance destination information as compared to character recognition.

The label may have a tab, and at least one of a location of the tab provided, a color of the tab, and a shape of the tab may be varied for each destination of conveyance. Thus, it is possible to reduce a situation in which the user picks up a wrong label and issues conveying instructions.

The label may be a label on which a character indicating the destination of conveyance is printed or a label in a color indicating the destination of conveyance. Thus, it is possible to reduce a situation in which the user picks up a wrong label and issues conveying instructions.

The wagon may include a label attachment portion that allows the label to be attached in a replaceable state. Thus, the user is able to easily issue conveying instructions.

The wagon may include a display device showing the conveyance destination information, and the acquisition unit may include a first sensor configured to detect the conveyance destination information from the display device or a communication unit configured to receive the conveyance destination information detected by a second sensor. With the thus configured control system, the user is able to issue conveying instructions with a simple method, and the transfer robot can receive the conveying instructions.

The wagon may include a display device showing the conveyance destination information, the acquisition unit may include a first camera configured to obtain image data by capturing an image on the display device or a communication unit configured to receive image data obtained by capturing an image on the display device with a second camera, and the controller may be configured to detect the conveyance destination information from the image data. With the thus configured control system, the user is able to issue conveying instructions with a simple method, and the transfer robot can receive the conveying instructions.

The wagon may include an RFID tag on which the conveyance destination information is recorded, and the acquisition unit may include a first RFID reader configured to read the conveyance destination information from the RFID tag or a communication unit configured to receive the conveyance destination information read from the RFID tag with a second RFID reader. With the thus configured control system, the user is able to issue conveying instructions with a simple method, and the transfer robot can accurately receive the conveying instructions.

The conveyance destination information may include information indicating a plurality of destinations of conveyance, and the controller may be configured to cause the transfer robot to autonomously move such that the transfer robot conveys the wagon along a route including the plurality of destinations of conveyance indicated by the conveyance destination information. With the thus configured control system, even when there is a plurality of destinations of conveyance, the transfer robot can convey all the articles to the designated destinations of conveyance.

The wagon may include a storage portion that accommodates the article and a supporting portion that supports the storage portion in a state where a space for allowing at least part of a chassis of the transfer robot to enter is provided under the storage portion, and the transfer robot may include a lifting and lowering mechanism that loads and unloads the wagon to or from a top surface side of the at least part of the chassis. With the thus configured control system, it is possible to easily automatically convey the wagon with the transfer robot.

A second aspect of the disclosure provides a control method. The control method includes: acquiring, by a control system that controls a conveyance system including a transfer robot capable of autonomously moving and capable of conveying an article to be conveyed, conveyance destination information indicating a destination of conveyance of the article from a wagon capable of accommodating the article; and causing the transfer robot, by the control system, to autonomously move such that the transfer robot conveys the wagon to the destination of conveyance indicated by the conveyance destination information. With the thus configured control method, when a user just causes the wagon to hold conveyance destination information in order to issue conveying instructions with a destination of conveyance of the article to the transfer robot, it is possible to acquire the conveyance destination information from the wagon and cause the transfer robot to autonomously move and convey the article to the destination of conveyance in accordance with the conveying instructions indicated by the conveyance destination information. Thus, with the control method, the user is able to easily issue conveying instructions with a designated destination of conveyance to the transfer robot.

The wagon may include a label on which the conveyance destination information is printed, and the control method may further include detecting, by a first sensor, the conveyance destination information from the label or receiving, by a communication unit, the conveyance destination information detected by a second sensor. With the thus configured control method, the user is able to easily issue conveying instructions without a device that needs electricity, and the transfer robot can receive the conveying instructions.

The wagon may include a label on which the conveyance destination information is printed, and the control method may further include obtaining, by a first camera, image data by capturing an image on the label or receiving, by a communication unit, image data obtained by capturing an image on the label with a second camera, and detecting the conveyance destination information from the image data. With the thus configured control method, the user is able to easily issue conveying instructions without a device that needs electricity, and the transfer robot can receive the conveying instructions.

The wagon may include a display device showing the conveyance destination information, and the control method may further include detecting, by a first sensor, the conveyance destination information or receiving, by a communication unit, the conveyance destination information detected by a second sensor. With the thus configured control method, the user is able to issue conveying instructions with a simple method, and the transfer robot can receive the conveying instructions.

The wagon may include a display device showing the conveyance destination information, and the control method may further include obtaining, by a first camera, image data by capturing an image on the display device or receiving, by a communication unit, image data obtained by capturing an image on the display device with a second camera, and detecting the conveyance destination information from the image data. With the thus configured control method, the user is able to issue conveying instructions with a simple method, and the transfer robot can receive the conveying instructions.

The wagon may include an RFID tag on which the conveyance destination information is recorded, and the control method may further include reading, by a first RFID reader, the conveyance destination information from the RFID tag or receiving, by a communication unit, the conveyance destination information read from the RFID tag with a second RFID reader. With the thus configured control method, the user is able to issue conveying instructions with a simple method, and the transfer robot can accurately receive the conveying instructions.

The conveyance destination information may include information indicating a plurality of destinations of conveyance, and the control method may further include causing the transfer robot to autonomously move such that the transfer robot conveys the wagon along a route including the plurality of destinations of conveyance indicated by the conveyance destination information. With the thus configured control method, even when there is a plurality of destinations of conveyance, the transfer robot can convey all the articles to the designated destinations of conveyance.

A third aspect of the disclosure provides a non-transitory storage medium. The non-transitory storage medium stores instructions executable on one or more processors of a computer of a control system that controls a conveyance system including a transfer robot capable of autonomously moving and capable of conveying an article to be conveyed. The instructions causes the one or more processors to execute functions. The functions include: (i) inputting conveyance destination information indicating a destination of conveyance of the article and acquired from a wagon capable of accommodating the article; and (ii) causing the transfer robot to autonomously move such that the transfer robot conveys the wagon to the destination of conveyance indicated by the input conveyance destination information. With the thus configured non-transitory storage medium, when a user just causes the wagon to hold conveyance destination information in order to issue conveying instructions with a destination of conveyance of the article to the transfer robot, it is possible to acquire the conveyance destination information from the wagon and cause the transfer robot to autonomously move and convey the article to the destination of conveyance in accordance with the conveying instructions indicated by the conveyance destination information. Thus, with the non-transitory storage medium, the user is able to easily issue conveying instructions with a designated destination of conveyance to the transfer robot.

The wagon may include a label on which the conveyance destination information is printed, and the conveyance destination information may be the conveyance destination information detected from the label by a first sensor or the conveyance destination information detected from the label by a second sensor and received by a communication unit. With the thus configured non-transitory storage medium, the user is able to easily issue conveying instructions without a device that needs electricity, and the transfer robot can receive the conveying instructions.

The wagon may include a label on which the conveyance destination information is printed, and the non-transitory storage medium may store instructions for causing the computer to detect the conveyance destination information from (i) image data obtained by capturing an image on the label with a first camera or (ii) image data obtained by capturing an image on the label with a second camera and receiving the image data from the second camera by a communication unit. With the thus configured non-transitory storage medium, the user is able to easily issue conveying instructions without a device that needs electricity, and the transfer robot can receive the conveying instructions.

The wagon may include a display device showing the conveyance destination information, and the conveyance destination information may be the conveyance destination information detected from the display device by a first sensor or the conveyance destination information detected from the display device by a second sensor and received by a communication unit. With the thus configured non-transitory storage medium, the user is able to issue conveying instructions with a simple method, and the transfer robot can receive the conveying instructions.

The wagon may include a display device showing the conveyance destination information, and the non-transitory storage medium may store instructions for causing the computer to detect the conveyance destination information from (i) image data obtained by capturing an image on the display device with a first camera or (ii) image data obtained by capturing an image on the display device with a second camera and receiving the image data from the second camera by a communication unit. With the thus configured non-transitory storage medium, the user is able to issue conveying instructions with a simple method, and the transfer robot can receive the conveying instructions.

The wagon may include an RFID tag on which the conveyance destination information is recorded, and the conveyance destination information may be the conveyance destination information read from the RFID tag with a first RFID reader or the conveyance destination information read with a second RFID reader and received by a communication unit. With the thus configured non-transitory storage medium, the user is able to issue conveying instructions with a simple method, and the transfer robot can accurately receive the conveying instructions.

The conveyance destination information may include information indicating a plurality of destinations of conveyance, and the non-transitory storage medium stores instructions for causing the transfer robot to autonomously move such that the transfer robot conveys the wagon along a route including the plurality of destinations of conveyance indicated by the input conveyance destination information. With the thus configured non-transitory storage medium, even when there is a plurality of destinations of conveyance, the transfer robot can convey all the articles to the designated destinations of conveyance.

According to the aspects of the disclosure, it is possible to provide a control system, a control method, and a non-transitory storage medium that allow a user to easily issue conveying instructions with a designated destination of conveyance to a transfer robot.

### BRIEF DESCRIPTION OF THE DRAWINGS

Features, advantages, and technical and industrial significance of exemplary embodiments of the disclosure will be described below with reference to the accompanying drawings, in which like signs denote like elements, and wherein:

FIG. 1 is a perspective view that shows an example of the overall configuration of a transfer robot according to an embodiment;

FIG. 2 is a perspective view that shows an example of the overall configuration of a wagon to be conveyed by the transfer robot of FIG. 1;

FIG. 3 is a table that illustrates a list of labels attachable to the wagon of FIG. 2;

FIG. 4 is a side view that shows a state where the transfer robot of FIG. 1 detects conveyance destination information from a label attached to the wagon of FIG. 2;

FIG. 5 is a perspective view that shows a state where the transfer robot of FIG. 1 is conveying the wagon of FIG. 2;

FIG. 6 is a front view that shows a state where the transfer robot of FIG. 1 is conveying the wagon of FIG. 2;

FIG. 7 is a side view that shows a state where the transfer robot of FIG. 1 is conveying the wagon of FIG. 2;

FIG. 8 is a flowchart for illustrating an example of a wagon conveying process that is executed by the transfer robot of FIG. 1;

FIG. 9 is a schematic diagram that shows an example of the wagon conveying process that is executed by the transfer robot of FIG. 1;

FIG. 10 is a schematic diagram that shows another example of the wagon conveying process that is executed by the transfer robot of FIG. 1;

FIG. 11 is a side view that shows a state where the transfer robot according to another configuration example of the embodiment reads conveyance destination information from an RFID tag mounted on a wagon;

FIG. 12 is a schematic diagram that shows an example of the overall configuration of a conveyance system including the transfer robot according to the embodiment;

FIG. 13 is a flowchart for illustrating an example of control over the transfer robot, which is executed by a host management apparatus in the conveyance system of FIG. 12; and

FIG. 14 is a diagram that shows an example of the hardware configuration of an apparatus.

### DETAILED DESCRIPTION OF EMBODIMENTS

Hereinafter, an embodiment of the disclosure will be described; however, the disclosure defined by the appended claims is not intended to be limited to the embodiment. All the components of the embodiment are not always indispensable.

### EMBODIMENT

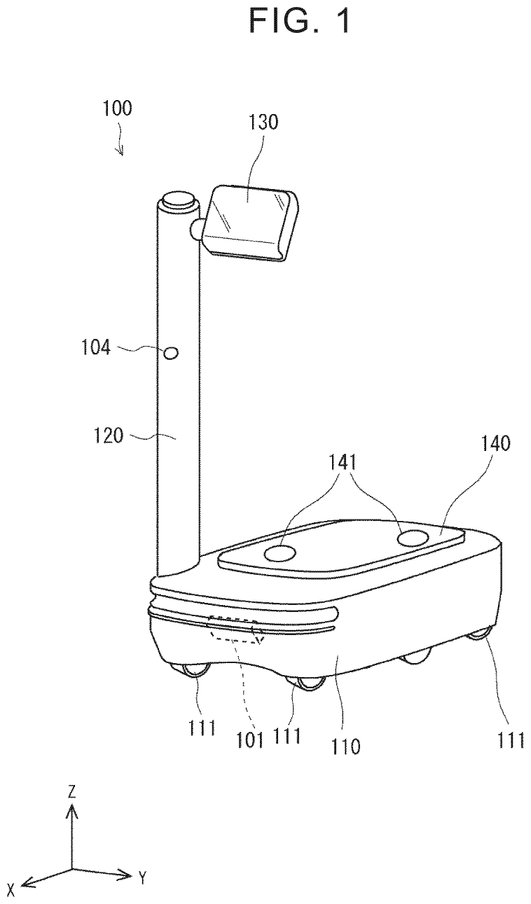

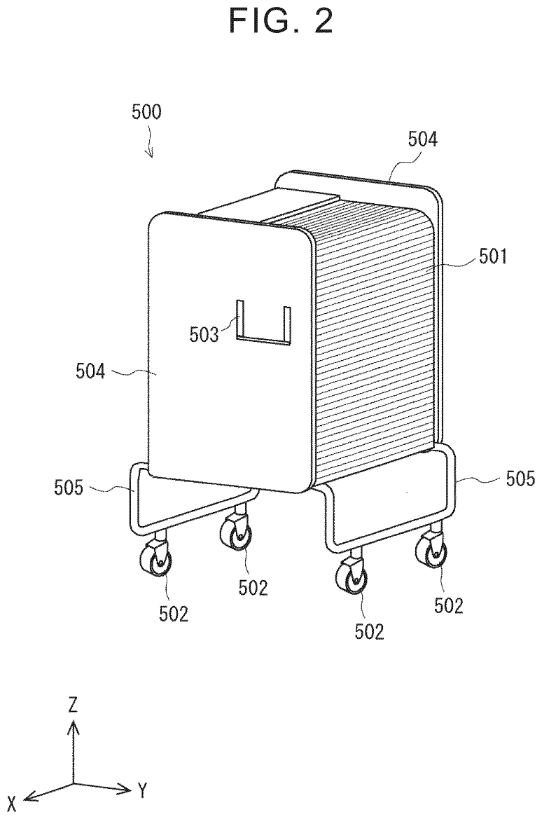

A control system according to the present embodiment controls a conveyance system including a transfer robot capable of autonomously moving and capable of conveying an article to be conveyed. The transfer robot is a mobile robot capable of conveying an article to be conveyed. Hereinafter, one configuration example of the transfer robot according to the present embodiment will be described with reference to FIG. 1 and FIG. 2. FIG. 1 is a perspective view that shows an example of the overall configuration of the transfer robot according to the present embodiment. FIG. 2 is a perspective view that shows an example of the overall configuration of a wagon to be conveyed by the transfer robot of FIG. 1.

The conveyance system just needs to include a transfer robot like the transfer robot 100 shown in FIG. 1. The conveyance system may include another apparatus, such as a host management apparatus. However, for the sake of simple description, initially, the major features of an example in which the conveyance system is made up of the transfer robot 100 alone will be described. In the case of this example, the control system may mean the transfer robot 100 itself or a component element of a control system of the transfer robot 100.

In the following description, an XYZ orthogonal coordinate system is used as needed. An X direction corresponds to a front and rear direction of the transfer robot 100 shown in FIG. 1, a Y direction corresponds to a right and left direction of the transfer robot 100, and a Z direction corresponds to a vertically up and down direction. More specifically, a positive X direction is defined as the forward direction of the transfer robot 100, and a negative X direction is defined as the backward direction of the transfer robot 100. A positive Y direction is a direction to the left-hand side of the transfer robot 100, and a negative Y direction is a direction to the right-hand side of the transfer robot 100. A positive Z direction is a vertically upward direction, and a negative Z direction is a vertically downward direction.

The transfer robot 100 is movable in both the forward direction and the backward direction. In other words, when wheels are rotated in the forward direction, the transfer robot 100 moves in the forward direction; when the wheels are rotated in the reverse direction, the transfer robot 100 moves in the backward direction. When the rotation speed of the right wheels and the rotation speed of the left wheels are varied, the transfer robot 100 can turn to the right and to the left.

As shown in FIG. 1, the transfer robot 100 may include a chassis 110 that loads an article to be conveyed, a stand 120, and an operating unit 130. Wheels 111, axles, a battery, a control computer 101, a drive motor, and the like are mounted in the chassis 110. The description will be made on the assumption that the control computer 101 is mounted at the location (shown in the drawing) of the chassis 110; however, the location is not limited thereto. The control computer 101 may be mounted at another location of the chassis 110 or part or whole of the control computer 101 may be mounted on at least one of the stand 120 and the operating unit 130.

The chassis 110 holds the wheels 111 such that the wheels 111 are rotatable. Various sensors, such as a camera and a distance measuring sensor, may be provided in the chassis 110 or the stand 120 to, for example, avoid contact with an obstacle and check a route. Although the transfer robot 100 is an autonomous mobile robot, the transfer robot 100 may have a function to be capable of moving in accordance with operation of a user, that is, may be a mobile robot capable of switching between autonomous moving mode and user operation mode. In control of the autonomous moving, the transfer robot can be caused to autonomously move by, for example, choosing a route or avoiding contact by using a learning model obtained through machine learning.

Here, the user operation mode in which the mobile robot moves based on user operation just needs to be relatively higher in the degree to which user operation is involved than the autonomous moving mode in which the mobile robot autonomously moves. In other words, the user operation mode does not need to be limited to a mode in which all the motions of the mobile robot are operated by the user and the entire autonomous control of the mobile robot is excluded. Similarly, the autonomous moving mode does not need to be limited to a mode in which the mobile robot executes completely autonomous control and operation of the user is not received at all. For example, the user operation mode and the autonomous moving mode may include the following first to third examples.

In the first example, in the autonomous moving mode, the mobile robot autonomously runs and determines whether to stop and whether to start running, and the user does not perform operation; in the user operation mode, the mobile robot autonomously runs, and the user performs operation to stop and operation to start running. In the second example, in the autonomous moving mode, the mobile robot autonomously runs, and the user performs operation to stop and operation to start running; in the user operation mode, the mobile robot does not autonomously run, and the user performs not only operation to stop and operation to start running but also operation to run. In the third example, in the autonomous moving mode, the mobile robot autonomously runs and determines whether to stop and whether to start running, and the user does not perform operation; in the user operation mode, the mobile robot autonomously runs for speed adjustment, contact avoidance, and the like, and the user performs operation to change a direction of running, a route, and the like.

The user may be a worker or the like in a facility where the transfer robot 100 is used, and may be a hospital worker when the facility is a hospital. The moving route or the like of the transfer robot 100 may be managed by a host management apparatus (not shown), and the transfer robot 100 may be configured to convey an article to be conveyed in accordance with instructions from the host management apparatus. In this case, an external apparatus (described later) may be the host management apparatus or a wireless communication device connected to the host management apparatus. Here, the host management apparatus is an example of a server capable of connecting with the transfer robot 100 by wireless communication. The server provides information for autonomous movement to the transfer robot 100. The server is not limited to a stand-alone apparatus and may be constructed as a system of which functions are distributed to multiple apparatuses.

The control computer 101 may be implemented by, for example, an integrated circuit. The control computer 101 may be implemented by, for example, a processor, such as a micro processing unit (MPU) and a central processing unit (CPU), a working memory, a nonvolatile storage device, and the like. When a program for control to be executed on the processor is stored in advance in the storage device and the processor loads the program onto the working memory and runs the program, it is possible to achieve the function of controlling the transfer robot 100. The control computer 101 may be referred to as a controller.

Although not shown in the drawing, the transfer robot 100 may include a wireless communication unit for wireless communication with the external apparatus. The wireless communication unit may be configured to perform wireless communication based on one or multiple wireless communication standards of various wireless communication standards, such as WiFi (registered trademark; hereinafter, the same applies) standard, with the external apparatus. The external apparatus may be, for example, a wireless local area network (LAN) access point, a repeater, or the like; however, the external apparatus is not limited thereto. The external apparatus just needs to be a wireless communication device or an apparatus with a wireless communication function.

The chassis 110 may include a lifting and lowering mechanism 140 that loads and unloads an article to be conveyed. Part of the lifting and lowering mechanism 140 is allowed to be accommodated inside the chassis 110. The lifting and lowering mechanism 140 can be disposed on the top surface side of the chassis 110 in a state where a mounting face that mounts an article to be conveyed is exposed. The lifting and lowering mechanism 140 is a lifting and lowering stage capable of moving up and down. The lifting and lowering mechanism 140 is capable of moving up and down in accordance with control from the control computer 101. The chassis 110 is provided with a motor and a guide mechanism that moves up and down. The top surface of the lifting and lowering mechanism 140 is a mounting face that mounts a wagon serving as an article to be conveyed. The wagon just needs to be a predetermined wagon with a size, a shape, and a weight such that the wagon can be mounted on the lifting and lowering mechanism 140 and conveyed. Hereinafter, a wagon 500 shown in FIG. 2 will be described as an example of the wagon; however, the wagon is not limited to the wagon 500. The lifting and lowering mechanism 140 includes a lift mechanism that lifts the wagon 500. A space on the upper side of the lifting and lowering mechanism 140 is a mounting space that mounts an article to be conveyed. Only when the user loads the wagon 500, the chassis 110 does not need to include the lifting and lowering mechanism 140.

The stand 120 is secured to the chassis 110. The stand 120 is a rod-shaped member extending upward from the chassis 110. Here, the stand 120 has a cylindrical columnar shape of which the longitudinal direction is the Z direction. Of course, the shape of the stand 120 is not limited, and the transfer robot 100 does not need to include the stand 120. The longitudinal direction of the stand 120 is provided parallel to the Z direction. The stand 120 is disposed outside the lifting and lowering mechanism 140. In other words, the stand 120 is disposed so as not to interfere with lifting and lowering operation of the lifting and lowering mechanism 140. The stand 120 is disposed at one end side of the chassis 110 in the Y direction (right and left direction). The stand 120 is secured near the right front corner of the chassis 110. In the XY-plane, the stand 120 is provided at the positive X-side and negative Y-side end of the chassis 110.

For example, a stick of a joystick apparatus may be provided at the top surface portion of the stand 120, or an emergency stop button, an indicator lamp indicating an operational status or the like of the transfer robot 100, or the like may be provided at the top surface portion of the stand 120. The joystick apparatus is used to, in the case of the user operation mode, perform operation to move the transfer robot 100 in a direction intended by the user.

The stand 120 supports the operating unit 130. The operating unit 130 is secured near the upper end of the stand 120. Thus, the operating unit 130 is allowed to be installed at the level at which the user can easily perform operation. In other words, the stand 120 extends to the level at which the user standing can easily perform operation. The operating unit 130 extends from the stand 120 toward the positive Y side. In the right and left direction, the operating unit 130 is disposed at the center of the chassis 110.

The operating unit 130 may include a touch panel monitor or the like which receives operation of the user. Of course, the operating unit 130 may include a microphone for voice input. The monitor of the operating unit 130 faces the opposite side from the chassis 110. In other words, a display surface (operating surface) of the operating unit 130 is a positive X-side surface. The operating unit 130 may be detachable from the stand 120. In other words, the stand 120 may include a holder that holds a touch panel. The user is able to input a destination of conveyance of an article to be conveyed, conveyance information on an article to be conveyed, or the like by operating the operating unit 130. In addition, the operating unit 130 can show, to the user, information, such as in-conveying, an article to be conveyed, details regarding an article scheduled to be conveyed, and its destination. Of course, the transfer robot 100 may be configured not to include the operating unit 130.

The stand 120 may include an acquisition unit that acquires conveyance destination information indicating a destination of conveyance of an article from the wagon 500 capable of accommodating the article. FIG. 1 shows an example in which a camera 104 faced toward the positive X side, which is an example of a sensor that detects conveyance destination information, is provided as the acquisition unit. The location of the sensor is not limited to the example in which the sensor is provided in the stand 120. The sensor may be provided in the chassis 110 or the operating unit 130. Various sensors that avoid contact with an obstacle, check a route, and the like may be used as the sensor.

The control computer 101 causes the transfer robot 100 to autonomously move such that the transfer robot 100 conveys the wagon 500 to the destination of conveyance indicated by the conveyance destination information detected by the sensor illustrated as the camera 104. The control computer 101 may be regarded as including a movement controller that executes control for such autonomous movement. Targets of autonomous movement control just need to include a drive unit (not shown) provided in the transfer robot 100. If the targets include the lifting and lowering mechanism 140, it is possible to automatically load the wagon 500 or load and unload an article. The drive unit may mean a portion that drives the wheels 111 and the like.

The user puts an article to be conveyed in the wagon 500 mounted on the transfer robot 100 and makes a request for conveyance. Hereinafter, the wagon 500 itself can also be referred to as an article to be conveyed, so, for the sake of convenience, an article to be conveyed, which is accommodated in the wagon 500, is referred to as an article and will be described as distinguished from each other. The transfer robot 100 autonomously moves to a set destination and conveys the wagon 500. In other words, the transfer robot 100 executes a task for conveying the wagon 500. In the following description, a place where the wagon 500 is loaded is referred to as a source of conveyance or a loading place, and a place to which the wagon 500 is delivered is referred to as a destination of conveyance or a destination.

For example, it is assumed that the transfer robot 100 moves in a general hospital with a plurality of diagnosis and treatment departments. The transfer robot 100 conveys an article, such as supplies, consumables, and medical tools, among the plurality of diagnosis and treatment departments. For example, the transfer robot 100 delivers an article from a nurse station of a diagnosis and treatment department to a nurse station of another diagnosis and treatment department. Alternatively, the transfer robot 100 delivers an article from a store cabinet for supplies and medical tools to a nurse station of a diagnosis and treatment department. The transfer robot 100 also delivers a medicine dispensed at a pharmacy to a diagnosis and treatment department or a patient that intends to use the medicine.

Examples of the article include a medicine, consumables, such as a bandage, a sample, an examination tool, a medical tool, a hospital diet, and supplies, such as stationery. Examples of medical instruments include a sphygmomanometer, a blood infusion pump, a syringe pump, a foot pump, a nurse call, a bed exit sensor, a continuous low-pressure inhaler, an electrocardiogramonitor, a drug infusion controller, an enteral nutrition pump, a ventilator, a cuff pressure meter, a touch sensor, an aspirator, a nebulizer, a pulse oximeter, a resuscitator, an aseptic device, and an echo device. The transfer robot 100 may convey a meal, such as a hospital diet and a diet for examination. The transfer robot 100 may convey a used instrument, an eating utensil used for a meal, or the like. When a destination of conveyance is on a different floor, the transfer robot 100 may move by using an elevator or the like.

The details of the wagon 500 of FIG. 2, an example in which conveyance destination information is held, an example in which the transfer robot 100 holds the wagon 500, and an example in which conveyance destination information is detected will be described below. The transfer robot 100 includes a sensor illustrated as the camera 104 as described above. Thus, the user just needs to cause the wagon 500 to hold conveyance destination information in order to issue conveying instructions with a destination of conveyance of an article to the transfer robot 100. In other words, only with such conveying instructions, the transfer robot 100 is capable of detecting conveyance destination information from the wagon 500 and autonomously moving and conveying an article to a destination of conveyance in accordance with the conveying instructions indicated by the conveyance destination information. Thus, with the transfer robot 100, the user is able to easily issue conveying instructions with a designated destination of conveyance to the transfer robot 100, that is, the user is able to easily issue conveying instructions with a designated destination of conveyance to the transfer robot 100.

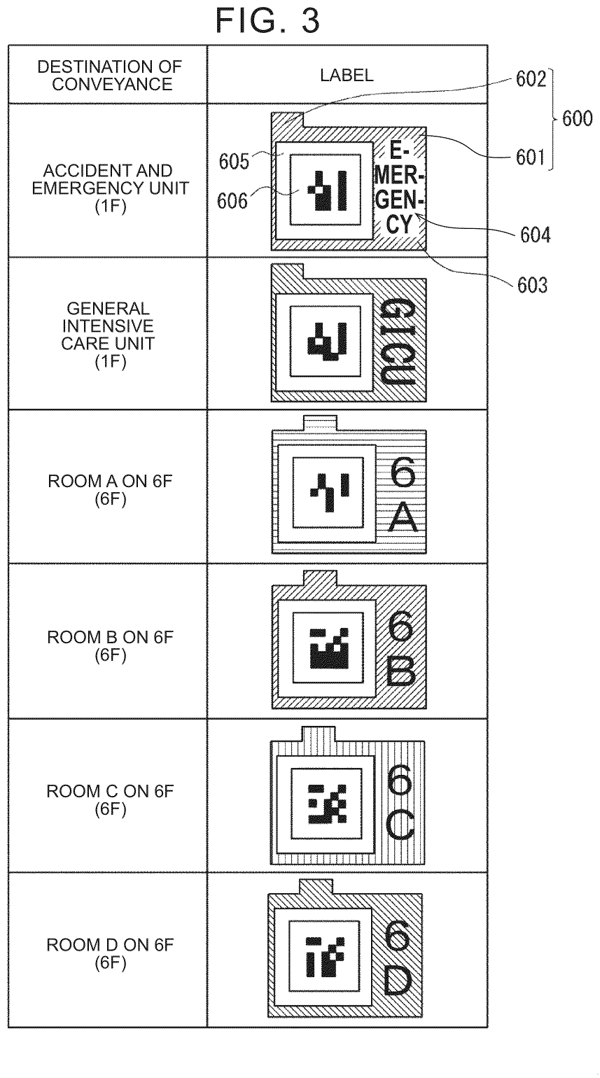

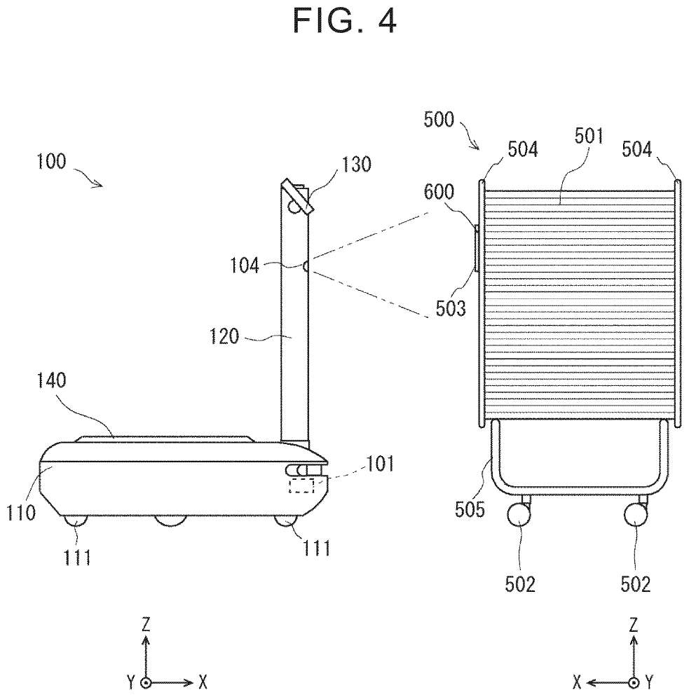

Next, the details of the wagon 500, an example in which conveyance destination information is held, an example in which the transfer robot 100 holds the wagon 500, and an example in which conveyance destination information is detected will be described with reference to FIG. 2 to FIG. 4. FIG. 3 is a table that illustrates a list of labels attachable to the wagon 500. FIG. 4 is a side view that shows a state where the transfer robot 100 detects conveyance destination information from a label 600 attached to the wagon 500.

The wagon 500 includes a storage portion for accommodating an article and a supporting portion that supports the storage portion in a state where a space for allowing at least part of the chassis 110 to enter is provided under the storage portion. As shown in FIG. 2, the storage portion may be configured to include side plates 504 respectively at both sides of the wagon 500 and an openable cover 501 When the user opens the cover 501, it is possible to load an article to be accommodated in the wagon 500 and unload an article accommodated in the wagon 500. As shown in FIG. 2, the supporting portion may be configured to include a support frame 505 that supports the storage portion and wheels 502 attached to the lower side of the support frame 505. Each of the wheels 502 may include a cover (not shown).

The wagon 500 may be held by the lifting and lowering mechanism 140 in the transfer robot 100 as described above. The lifting and lowering mechanism 140 is a mechanism that loads the wagon 500 serving as an article to be conveyed to the top surface side of at least part of the chassis 110 and unloading the wagon 500 from the top surface side. When the transfer robot 100 includes the lifting and lowering mechanism 140, it is possible to easily automatically convey the wagon 500.

The wagon 500 may include a label attachment portion 503 that allows a label 600 as illustrated in FIG. 3 to be attached in a replaceable state. The label 600 is used to detect conveyance destination information with the camera 104. The label 600 corresponds to a destination of conveyance selected by the user in the list shown in FIG. 3. As shown in FIG. 2, the label attachment portion 503 may be provided on one of the side plates 504; however, the location of the label attachment portion 503 is not limited. For example, the label attachment portion 503 may be provided on the cover 501. When the label attachment portion 503 is provided in the wagon 500, the user is able to easily issue conveying instructions.

However, when, for example, at least part of the side plate 504 or the cover 501 is made of a ferromagnetic substance, such as iron, and at least part of the label 600 is a magnet as well, it is possible to easily issue conveying instructions. Of course, when, for example, at least part of the side plate 504 or the cover 501 is a magnet and at least part of the label 600 is made of a ferromagnetic substance, such as iron, as well, a similar advantageous effect is obtained. Alternatively, when a repeatedly usable adhesive substance is applied to the back side of the label 600 or a repeatedly usable double-side adhesive tape is affixed to the back side of the label 600 as well, it is possible to easily issue conveying instructions. In this case, when a sticking location is determined at a predetermined place of the side plate 504 or the cover 501 and the label 600 is displayed, a place where the camera 104 reads is determined, so it is easy to detect conveyance destination information. Other than that, conveying instructions may be issued by putting the label 600 in a holder, such as a transparent bag, provided at a predetermined place of the side plate 504 or the cover 501.

A two-dimensional code indicating a destination of conveyance may be printed on the label 600 as conveyance destination information. The two-dimensional code may be a matrix two-dimensional code like a QR code (registered trademark; hereinafter, the same applies) 606; however, the two-dimensional code is not limited thereto. When such a two-dimensional code is printed in advance on the label 600, it is possible to reduce wrong recognition of conveyance destination information by the transfer robot 100 as compared to when characters are detected and recognized. A region 605 is a region in a color different from a color of a body 601 around the QR code 606. The region 605 is provided to increase the accuracy that the camera 104 reads the QR code 606. Of course, instead of a two-dimensional code, a one-dimensional code, such as a barcode, a special character, a special pattern, or the like may be printed as conveyance destination information.

The label 600 may include the body 601 and a tab as illustrated in the label for each of the destinations of conveyance of FIG. 3, and the materials may be any material except an example in which the label 600 is made of a magnet or a ferromagnetic substance or an example in which an adhesive substance is used for the label 600. At least one of a location indicating a destination of conveyance, a color indicating a destination of conveyance, and a shape indicating a destination of conveyance may be varied and provided for the tab 602. Thus, it is possible to reduce a situation in which the user picks up the wrong label 600 and issues conveying instructions. In FIG. 3, the location at which the tab 602 is provided is varied between a destination of conveyance on IF and a destination of conveyance on 6F. In other words, FIG. 3 shows an example in which the tab 602 is disposed at a location indicating a floor to which a destination of conveyance belongs in a service facility as the location of a destination of conveyance with respect to the body 601. Of course, instead of the label 600, a label with no tab 602 may be used.

The label 600 may be a label on which characters indicating a destination of conveyance are printed. For example, as shown in FIG. 3, character information 604 indicating a destination of conveyance may be printed on the body 601 as conveyance destination information. In other words, the character information 604 may include at least one of a name of a destination of conveyance and a symbol indicating a destination of conveyance. Thus, it is possible to reduce a situation in which the user picks up a wrong label 600 and issues conveying instructions. In this example as well, the label 600 may include the tab 602, and the same characters as the characters indicated by the character information 604 may be additionally printed on the tab 602 although not shown in the drawing.

The label 600 may be a label in a color indicating a destination of conveyance. For example, the body 601 may be in a color indicating a destination of conveyance as conveyance destination information such that a difference in color 603 between the label of an accident and emergency unit and the label of a general intensive care unit in FIG. 3 is indicated by a difference in hatching. Thus, it is possible to reduce a situation in which the user picks up a wrong label 600 and issues conveying instructions. In this example as well, the label 600 may include the tab 602, and the color of the tab 602 may be the same as the color of the body 601.

In the example of FIG. 3, an example in which the same color 603 is used for different destinations of conveyance, for example, an accident and emergency unit and room B on 6F is shown. This is an example on the assumption that a destination of conveyance is distinguished by the QR code 606 or the character information 604. In the example of FIG. 3, it is possible to discriminate another type, such as discrimination in emergency and discrimination in person in charge, such as an attending doctor and an attending nurse, in accordance with a difference in color. Therefore, it is further easy for the user to understand information on a destination of conveyance, with the result that it is possible to reduce wrong conveying instructions.

Conveyance destination information may be printed on one side of the label 600, and moving destination information indicating a destination of movement to which the wagon 500 is moved after being conveyed to the destination of conveyance indicated by the conveyance destination information may be printed on the other side of the label 600. Thus, the user is able to issue instructions to return the wagon 500 with simple work, that is, just by turning the label 600 upside down.

Various printing contents and printing locations described above in relation to the label 600 may be adopted solely or in combination. Printing of various pieces of information may be performed on a peel-off sticker. In this case, the peel-off sticker is affixed to the label 600. Since the label 600 is a label indicating a destination of conveyance, the label 600 may be referred to as a conveyance destination label or a destination indicating label.

As shown in FIG. 4, in the transfer robot 100, the camera 104 is capable of capturing an image on the label 600 in a state of being faced to the label 600 of the wagon 500, and the control computer 101 can detect conveyance destination information from image data obtained by capturing the image on the label 600 with the camera 104. In other words, in this example, the user is able to issue conveying instructions including a destination of conveyance with only the label 600, and the transfer robot 100 can recognize the destination of conveyance. Particularly, in such service using the label 600, the user is able to easily issue conveying instructions without a device that needs electricity, and the transfer robot 100 can receive the conveying instructions. The user is able to easily visually recognize a destination of conveyance with characters printed on the label 600 and the shape, color, and the like of the label 600, so it is possible to reduce wrong conveying instructions. Particularly, in the case of service in a hospital, the user is able to issue conveying instructions without touching the operating unit 130 or another area of the transfer robot 100, so the advantageous effect of infection control of communicable disease and the like is obtained, and the advantageous effect that a medical staff who often performs operation with gloves is able to easily issue instructions for a destination of conveyance.

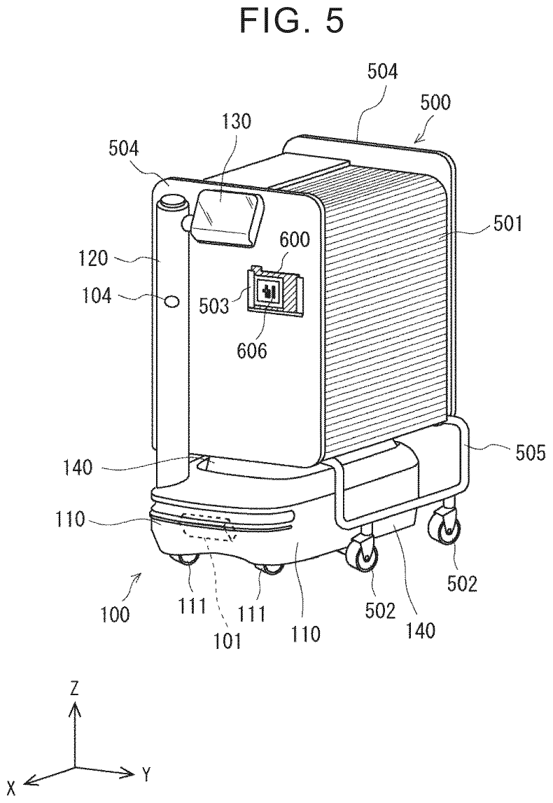

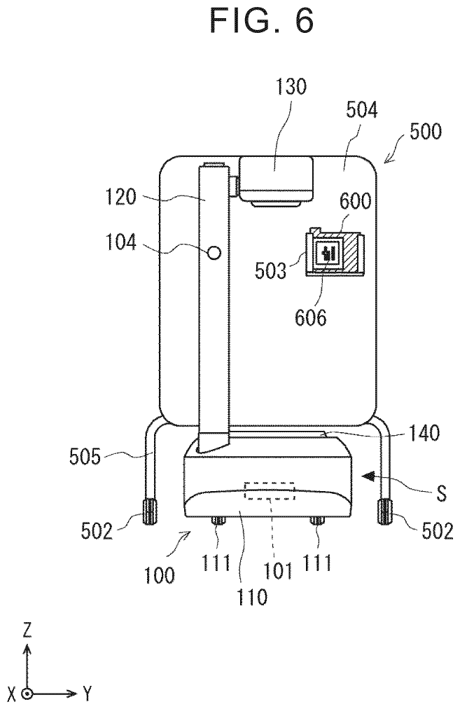

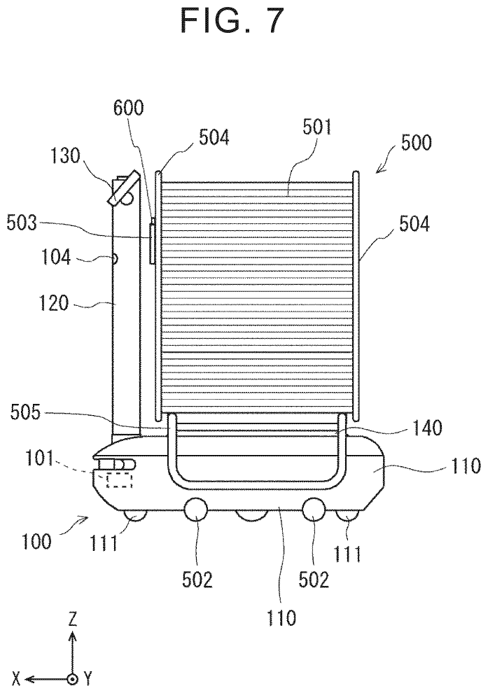

Next, a state where the transfer robot 100 conveys the wagon 500 serving as an article to be conveyed will be described with reference to FIG. 5 to FIG. 7. FIG. 5 is a perspective view that shows a state where the transfer robot 100 of FIG. 1 is conveying the wagon 500 of FIG. 2. FIG. 6 is a front view that shows the state. FIG. 7 is a side view that shows the state.

The transfer robot 100 can hold the wagon 500 with the lifting and lowering mechanism 140 as shown in FIG. 5 to FIG. 7. A space that allows at least part of the chassis 110 to enter is a space S provided under the wagon 500 as shown in FIG. 6, and the space S is a space that the chassis 110 enters. In other words, the chassis 110 is allowed to enter the space S just below the wagon 500. When the chassis 110 loads the wagon 500, the transfer robot 100 moves in the negative X direction to enter just below the wagon 500. The chassis 110 enters just below the wagon 500 from the side where the stand 120 is not provided in the front and rear direction. With this configuration, the stand 120 does not interfere with the wagon 500, and the wagon 500 can be loaded. In other words, the stand 120 is secured near the corner of the chassis 110 so as not to interfere with the wagon 500.

The camera 104 may be provided on the side surface of the stand 120 on the negative X side instead of the side surface of the stand 120 on the positive X side, shown in the drawing. Thus, on the assumption that the transfer robot 100 conveys the wagon 500, the transfer robot 100 can enter in the above-described entry direction before detecting conveyance destination information and then can detect the conveyance destination information printed on the label 600 with the camera 104 in the course of entry.

As shown in FIG. 1, a mounting surface of the lifting and lowering mechanism 140 may have a recess 141. On the other hand, a projection (not shown) may be provided on the lower side of the storage portion of the wagon 500. When the projection is fitted to the recess 141, the wagon 500 is fixed to the transfer robot 100.

The wagon 500 is illustrated as a carriage having the wheels 502; however, the shape and configuration of the wagon 500 are not limited. A predetermined wagon illustrated as the wagon 500 just needs to have a shape, a size, and a weight such that the wagon can be conveyed by the transfer robot 100.

An operation to load the wagon 500 will be described. When the chassis 110 enters the space S just below the wagon 500, the lifting and lowering mechanism 140 moves up. Thus, the lifting and lowering stage that is the top surface of the lifting and lowering mechanism 140 contacts with the wagon 500. The lifting and lowering mechanism 140 is capable of lifting the wagon 500. In other words, when the lifting and lowering mechanism 140 moves up, the wheels 502 leave the ground, and the wagon 500 is loaded on the chassis 110.

When the wagon 500 is unloaded from the chassis 110, the lifting and lowering mechanism 140 moves down. The wheels 502 contact with a floor surface, and the top surface of the lifting and lowering mechanism 140 leaves the wagon 500, The wagon 500 is placed on the floor surface. The wagon 500 is allowed to be unloaded from the chassis 110. The chassis 110 is provided with the four wheels 111. The four wheels 111 include right and left front wheels and right and left rear wheels. When the rotation direction and rotation speed of each of the wheels 111 are controlled independently of one another, the transfer robot 100 moves along a desired route. One or some of the four wheels 111 may be drive wheels, and the remaining wheels may be driven wheels. As shown in FIG. 1 and the like, an additional driven wheel may be provided, for example, between the front and rear wheels 111.

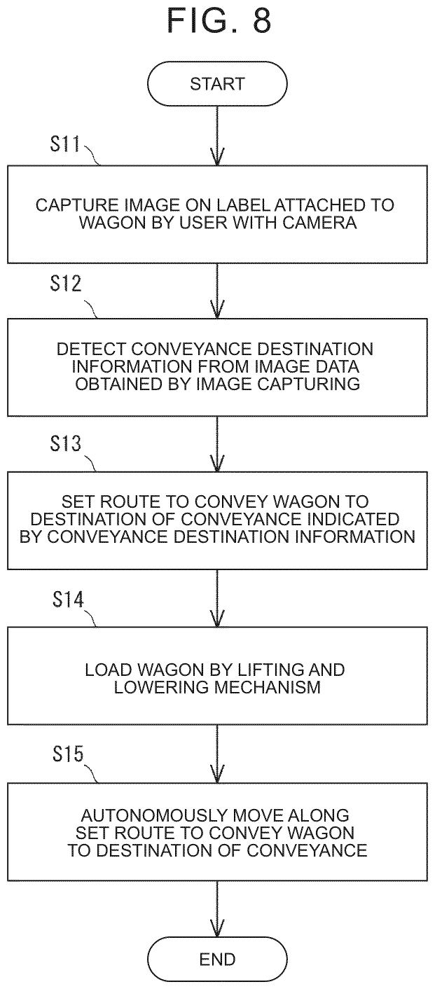

Next, an example of a wagon conveying process that is executed by the transfer robot 100 will be described with reference to FIG. 8. FIG. 8 is a flowchart for illustrating an example of the wagon conveying process that is executed by the transfer robot 100.

Initially, the user attaches the label 600 at the label attachment portion 503 and moves the wagon 500 to a predetermined place or the user goes to a place where the wagon 500 is placed and attaches the label 600 at the label attachment portion 503. As a result, the user is able to issue conveying instructions to the transfer robot 100 that reads the label 600. After that, as will be described below, the transfer robot 100 picks up the wagon 500 and conveys the wagon 500 to a destination of conveyance.

The transfer robot 100 recognizes the wagon 500 with the camera 104 or another sensor, and the camera 104 captures an image on the label 600 attached to the wagon 500 by the user (step S11). Subsequently, the control computer 101 detects conveyance destination information from image data obtained by image capturing (step S12). Thus, the control computer 101 recognizes a destination of conveyance, that is, a destination. Subsequently, the control computer 101 sets a route to convey the wagon 500 to the destination of conveyance indicated by the conveyance destination information (step S13). Setting of step S13 may be executed in cooperation with a host management apparatus.

After step S13, the control computer 101 executes control to load the wagon 500 with the lifting and lowering mechanism 140, that is, control to pick up the wagon 500 (step S14). Thus, the transfer robot 100 docks with the wagon 500 to complete preparations to move toward the destination of conveyance. Here, when the label 600 is attached to the wagon 500, a person does not need to remove or invalidate the label 600 during docking. Control of step S14 may be executed after step S12 and before step S13 or may be executed in parallel with step S13. Subsequently, the control computer 101 controls driving of the wheels 111 and the like such that the transfer robot 100 autonomously moves along the set route, the transfer robot 100 conveys the wagon 500 to the destination of conveyance (step S15), and the process ends.

In relation to step S11, the transfer robot 100 may be set in advance as a target to convey the wagon 500 and may be a transfer robot that moves looking for the wagon 500 or moves to a known location. For example, a host management apparatus may designate the wagon 500 of which the location is designated as a target to be conveyed or may designate the wagon 500 of which the location is designated as a search target, and the transfer robot 100 may autonomously move for conveying the wagon 500. Alternatively, when the transfer robot 100 finds the wagon 500 in a return route or the like after a conveying task for conveying another wagon or article is complete, the transfer robot 100 may automatically convey the wagon 500 to a destination of conveyance. Not limited to these examples, various methods are applicable as an operation method for conveying the wagon 500 with the transfer robot 100.

In each of the above-described various examples, the description has been made on the assumption that the transfer robot 100 conveys a wagon like the wagon 500 as an article to be conveyed. However, the transfer robot 100 just needs to be capable of conveying a wagon and may convey an individual article (package) as an article to be conveyed in operation. In such a case, a storage box, a shelf, or the like with which an article does not fall during moving may be mounted on the transfer robot 100.

In operation, there can be a case where the transfer robot 100 conveys a plurality of articles and the articles need to be conveyed to a plurality of destinations of conveyance. In this case, irrespective of whether the wagon 500 is used for conveyance, the user is able to unload an article at a destination of conveyance. The transfer robot 100 may autonomously move to a set destination or move in accordance with user operation and convey a wagon or an individual article.

However, in the above-described case, conveyance destination information should not include only information indicating a single destination of conveyance should include information indicating a plurality of destinations of conveyance. In other words, when a plurality of articles of different destinations of conveyance is accommodated in the wagon 500, conveyance destination information should include information indicating all the destinations of conveyance for completing conveyance of all the articles accommodated. For example, types of the number of destinations of conveyance that can be set during operation may be indicated on the label 600 with one or multiple indication methods, including a two-dimensional code, a one-dimensional code, a special character, a special pattern, and the like.

The control computer 101 just needs to cause the transfer robot 100 to autonomously move such that the transfer robot 100 conveys the wagon 500 along a route including the plurality of destinations of conveyance indicated by the conveyance destination information detected with the camera 104. In this case, the transfer robot 100 executes a conveying task for conveying a wagon, and the conveying task is a conveying task to pass through the plurality of destinations of conveyance and to make it possible to deliver all the articles accommodated. Calculation of this route can also be executed solely by the control computer 101 or may also be executed in cooperation with a host management apparatus. With such a configuration, in a case where there is a plurality of destinations of conveyance to convey all the articles in the wagon 500, the transfer robot 100 can convey all the articles to the designated destinations of conveyance.

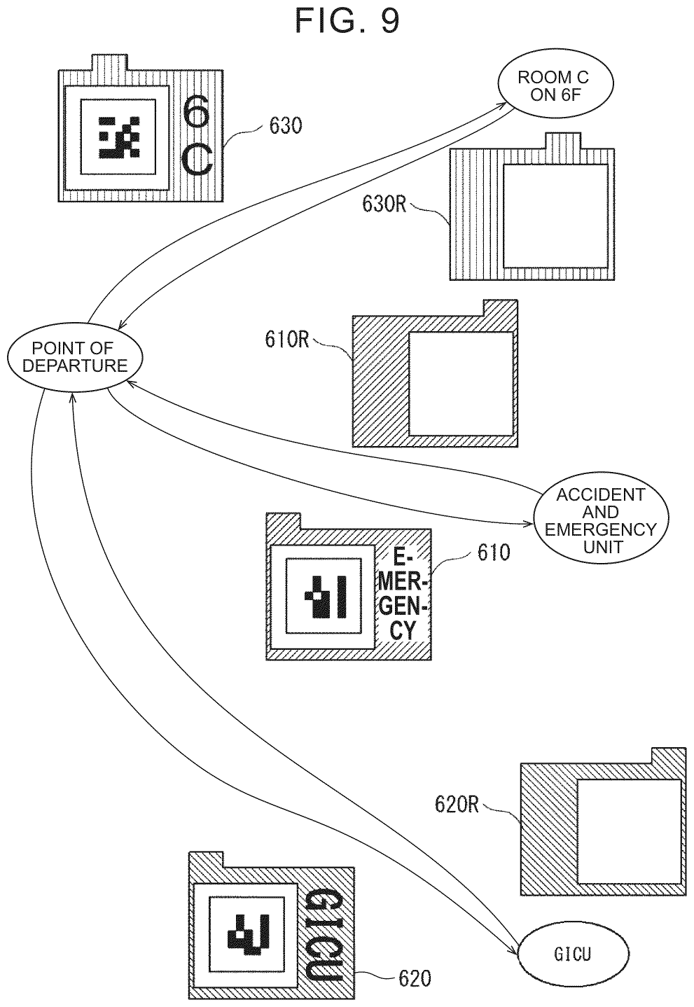

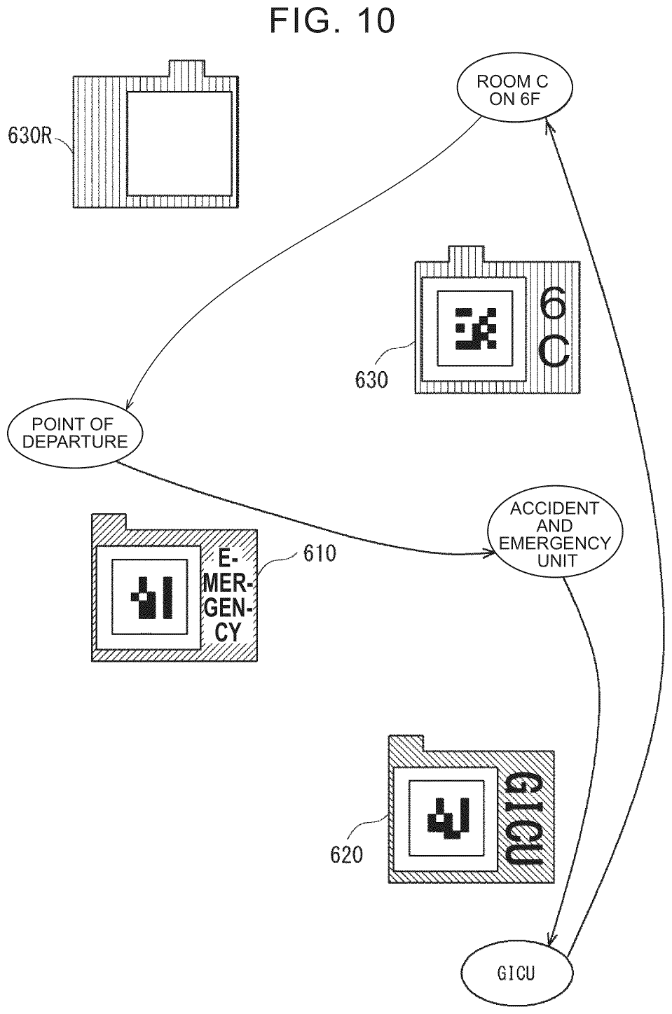

An example of the wagon conveying process in the case of a single destination of conveyance and an example of the wagon conveying process in the case of a plurality of destinations of conveyance will be described with reference to FIG. 9 and FIG. 10. FIG. 9 is a schematic diagram that shows an example of the wagon conveying process that is executed by the transfer robot 100. FIG. 10 is a schematic diagram that shows another example of the wagon conveying process that is executed by the transfer robot 100.

FIG. 9 shows an example of the case where a label 610 is attached to the wagon 500, an example of the case where a label 620 is attached to the wagon 500, and an example of the case where a label 630 is attached to the wagon 500. When the label 610 is attached to the wagon 500, the transfer robot 100 moves from a point of departure to an accident and emergency unit in accordance with the label 610 indicating the accident and emergency unit as a destination of conveyance. After that, when a hospital staff turns the label 610 upside down into a label 610R, the transfer robot 100 is allowed to return to the point of departure. Here, a part corresponding to a QR code is plain on the label 610R, and FIG. 9 shows an example in which, when the part corresponding to a QR code is plain, the destination of conveyance is recognized as the point of departure. Of course, the entire back side of the label 610 may be plain. The same applies to the labels 620, 630.

Similarly, when the label 620 is attached to the wagon 500, the transfer robot 100 moves from the point of departure to a general intensive care unit in accordance with the label 620 indicating the general intensive care unit as a destination of conveyance. After that, when a hospital staff turns the label 620 upside down into a label 620R, the transfer robot 100 is allowed to return to the point of departure. Similarly, when the label 630 is attached to the wagon 500, the transfer robot 100 moves from the point of departure to room C on the sixth floor in accordance with the label 630 indicating room C on the sixth floor as a destination of conveyance. After that, when a hospital staff turns the label 630 upside down into a label 630R, the transfer robot 100 is allowed to return to the point of departure.

When both sides of the label 600 are used as in the case of the example, application of adhesive substance or affixation of double-side adhesive tape just needs to be applied to both sides and not only to the back side so that the label 600 can be affixed even when turned upside down.

FIG. 10 shows an example in which a label 610, a label 620, and a label 630 are attached to the wagon 500 so that the label 610, the label 620, and the label 630 can be seen from outside in order from above or attached to the wagon 500 so that the label 610, the label 620, and the label 630 can be seen from outside in order from the left-hand side or the right-hand side.

The transfer robot 100 initially moves from a point of departure to an accident and emergency unit in accordance with the label 610 indicating the accident and emergency unit as a destination of conveyance. After that, the transfer robot 100 moves from the accident and emergency unit to a general intensive care unit in accordance with the label 620 indicating the general intensive care unit as a destination of conveyance. After that, the transfer robot 100 moves from the general intensive care unit to room C on the sixth floor in accordance with the label 630 indicating room C on the sixth floor as a destination of conveyance. After that, when a hospital staff turns the label 630 upside down into a label 630R, the transfer robot 100 is allowed to return to the point of departure. Instead of turning the label 630 upside down, a plain label for the point of departure may be attached to a location in order subsequent to the label 630 on the wagon 500.

In this way, the transfer robot 100 is capable of conveying the wagon 500 sequentially from the point of departure while going around a ward, and a hospital staff is able to take out a necessary article from the wagon 500 at each destination of conveyance. When, for example, the cover 501 of the transfer robot 100 is opened and then closed or when the transfer robot 100 detects operation to allow the transfer robot 100 to go to the next destination of conveyance, the transfer robot 100 may determine that an article has been taken out and go toward the next destination of conveyance. When, for example, the same article is conveyed along such a route, a hospital staff just needs to take out the article, use the article, and return the article at each destination of conveyance. When the cover 501 of the transfer robot 100 is opened and then closed or when the transfer robot 100 detects operation to allow the transfer robot 100 to go to the next destination of conveyance, the transfer robot 100 is allowed to go toward the next destination of conveyance.

FIG. 10 shows an example in which the label 600 is prepared for each destination of conveyance as an example of the wagon conveying process in a case where there is a plurality of destinations of conveyance. However, if a case of conveyance along the same route frequently occurs, a plurality of pieces of conveyance destination information respectively indicating destinations of conveyance may be indicated on one label 600 in advance. In this case, a plurality of pieces of conveyance destination information may be indicated such that the order of destinations of conveyance is indicated by predetermined order, such as order from above in the label 600. When the transfer robot 100 can set the next destination of conveyance at a point in time of arrival at a first destination of conveyance.

In relation to the acquisition unit, to acquire conveyance destination information indicated by a label, the transfer robot 100 may include a communication unit made up of a communication interface or the like for communication with a camera that obtains image data by capturing an image on the label, instead of the camera 104. The camera may be disposed at one or multiple locations in a facility where the transfer robot 100 is in service and may be referred to as an environmental camera. The communication unit just needs to be capable of receiving image data from the environmental camera connected to the transfer robot 100. For example, in the case of a configuration that the environmental camera is connected to a host management apparatus, the communication unit is capable of receiving image data from the host management apparatus. In this case, the transfer robot 100 may be configured to detect conveyance destination information by analyzing the received image data.

Alternatively, to acquire conveyance destination information indicated by a label, the transfer robot 100 may include a sensor of another type instead of the camera 104, and the sensor just needs to be the one capable of detecting conveyance destination information from the label. Alternatively, to acquire conveyance destination information indicated by a label, the transfer robot 100 may include a communication unit made up of a communication interface or the like that communicates with a sensor that detects conveyance destination information from the label, instead of the camera 104. The sensor may be disposed at one or multiple locations in a facility where the transfer robot 100 is in service and may be referred to as an environmental sensor. The communication unit just needs to be capable of receiving conveyance destination information from the environmental sensor connected to the transfer robot 100. For example, in the case of a configuration that the environmental sensor is connected to a host management apparatus, the communication unit is capable of receiving conveyance destination information from the host management apparatus. The environmental sensor may be an environmental camera. In other words, to acquire conveyance destination information indicated by a label, the transfer robot 100 may include a communication unit made up of a communication interface or the like for communication with an environmental camera that detects conveyance destination information, instead of the camera 104. The communication unit just needs to be capable of receiving conveyance destination information detected from the environmental camera connected to the transfer robot 100. For example, in the case of a configuration that the environmental camera is connected to a host management apparatus, the communication unit is capable of receiving conveyance destination information from the host management apparatus.

The wagon 500 may include a display device showing conveyance destination information, instead of the label 600. A display device, such as a liquid crystal display and an organic electroluminescence display, may be used as the display device. Therefore, a tablet terminal, a smartphone, or the like, provided with such a display device may also be used. A presentation medium that does not need or less needs electric power to hold an image and that needs small electric power at the time of rewriting the image, such as electronic paper, may be used as the display device. Such a presentation medium can be easily handled in terms of a thin shape and a light weight. In this way, when the display device is provided instead of the label 600, the camera 104 just needs to capture an image on the display device, and the control computer 101 just needs to detect conveyance destination information from image data obtained by capturing the image on the display device with the camera 104. The other points are similar to those of the example in which the label 600 is adopted, so the description is omitted. With the thus configured transfer robot 100, the user is able to issue conveying instructions with a simple method, and the transfer robot 100 can receive the conveying instructions. When the display device is adopted, the wagon 500 may include a display device attachment portion similar to the label attachment portion 503.

To acquire conveyance destination information indicated by the display device, the transfer robot 100 may include a communication unit made up of a communication interface or the like for communication with a camera that obtains image data by capturing an image on the display device, instead of the camera 104. The camera may be disposed at one or multiple locations in a facility where the transfer robot 100 is in service and may be referred to as an environmental camera. The communication unit just needs to be capable of receiving image data from the environmental camera connected to the transfer robot 100. For example, in the case of a configuration that the environmental camera is connected to a host management apparatus, the communication unit is capable of receiving image data from the host management apparatus. In this case, the transfer robot 100 may be configured to detect conveyance destination information by analyzing the received image data.

Alternatively, to acquire conveyance destination information indicated by a display device, the transfer robot 100 may include a sensor of another type instead of the camera 104, and the sensor just needs to be the one capable of detecting conveyance destination information from the display device. Alternatively, to acquire conveyance destination information indicated by a display device, the transfer robot 100 may include a communication unit made up of a communication interface or the like for communication with a sensor that detects conveyance destination information from the display device, instead of the camera 104. The sensor may be disposed at one or multiple locations in a facility where the transfer robot 100 is in service and may be referred to as an environmental sensor. The communication unit just needs to be capable of receiving conveyance destination information from the environmental sensor connected to the transfer robot 100.

For example, in the case of a configuration that the environmental sensor is connected to a host management apparatus, the communication unit is capable of receiving conveyance destination information from the host management apparatus. The environmental sensor may be an environmental camera. In other words, to acquire conveyance destination information indicated by a display device, the transfer robot 100 may include a communication unit made up of a communication interface or the like for communication with an environmental camera that detects conveyance destination information, instead of the camera 104. The communication unit just needs to be capable of receiving conveyance destination information detected from the environmental camera connected to the transfer robot 100. For example, in the case of a configuration that the environmental camera is connected to a host management apparatus, the communication unit is capable of receiving conveyance destination information from the host management apparatus.

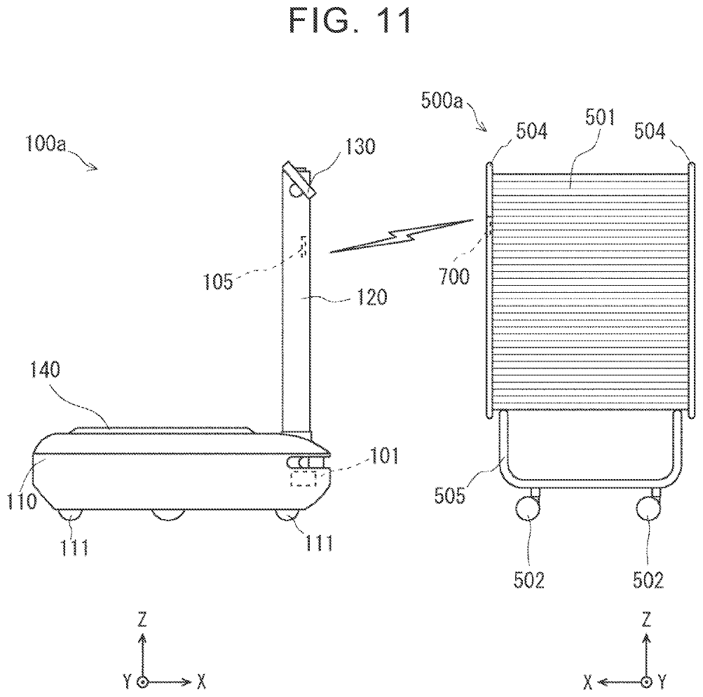

The wagon 500 may use an RFID tag instead of the label 600. A configuration example of a transfer robot that supports an RFID tag will be described with reference to FIG. 11. FIG. 11 is a side view that shows a state where the transfer robot according to another configuration example of the present embodiment reads conveyance destination information from an RFID tag mounted on a wagon.

The wagon 500a differs from the wagon 500 in that an RFID tag 700 on which conveyance destination information is recorded is provided instead of the label 600. In this case, the transfer robot 100a may include an RFID reader 105 that reads conveyance destination information from the RFID tag 700, instead of the camera 104, as a sensor. The other points are similar to those of the example of the wagon 500 and the transfer robot 100, so the description is omitted. With the transfer robot 100a, the user is able to issue conveying instructions with a simple method, and the transfer robot 100a can accurately receive the conveying instructions.

The wagon 500a may include a tag attachment portion (not shown) at which the RFID tag 700 is attached in a replaceable state and which is similar to the label attachment portion 503. Thus, it is possible to associate conveyance destination information corresponding to the wagon 500a, with the wagon 500a by replacing the RFID tag 700 instead of rewriting RFID. However, in this case, the user performs replacement, so character information indicating conveyance destination information may be printed on the RFID tag 700 or a peel-off sticker indicating character information may be affixed to the RFID tag 700.

To acquire conveyance destination information indicated by the RFID tag 700, the transfer robot 100a may include a communication unit made up of a communication interface or the like for communication with an RFID reader that reads conveyance destination information from the RFID tag 700, instead of the RFID reader 105. The communication unit just needs to be capable of receiving conveyance destination information from an RFID reader connected to the transfer robot 100a. For example, in the case of a configuration that the RFID reader is connected to a host management apparatus, the communication unit is capable of receiving conveyance destination information from the host management apparatus.

In the above description, the example in which the conveyance system is mainly made up of the transfer robot 100 has been described; however, the control system according to the present embodiment just needs to control the conveyance system as described above. The conveyance system may include a server capable of connecting with the transfer robot 100 by wireless communication. The server is a server that provides information for autonomous movement to the transfer robot 100. The server may be referred to as a host management apparatus since the server manages the transfer robot 100. The server is not limited to being made up of a stand-alone apparatus and may be constructed as a system of which functions are distributed to multiple apparatuses.

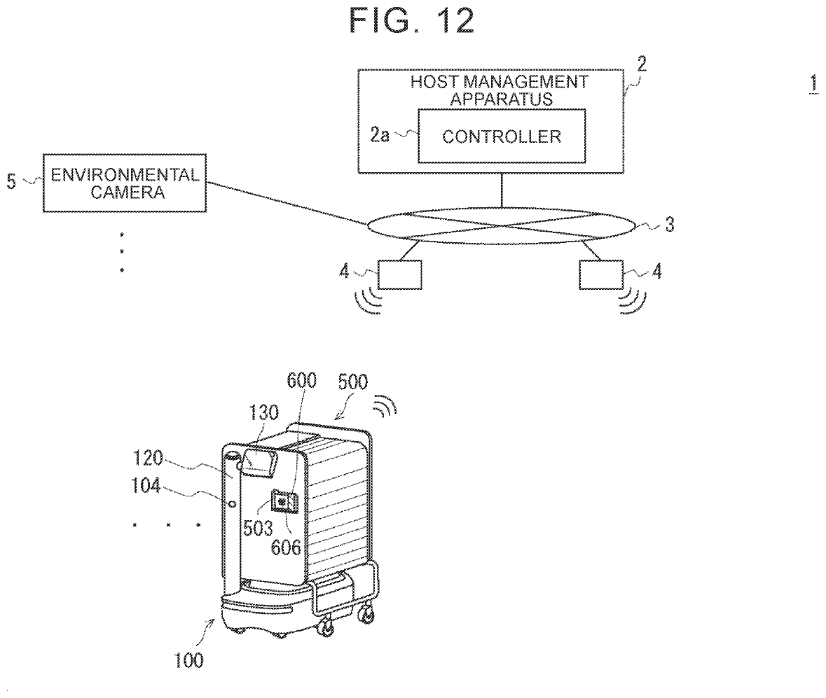

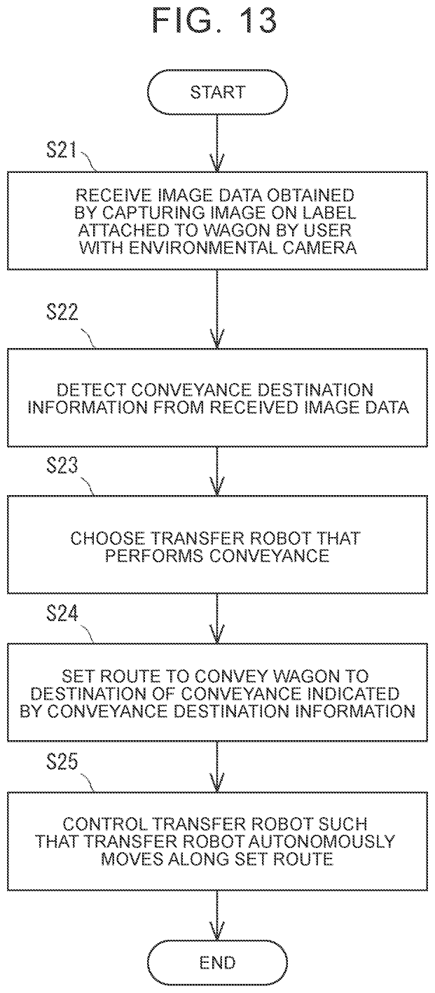

Hereinafter, an example in which the conveyance system includes the transfer robot 100, the host management apparatus, and the wagon 500 and conveyance destination information is obtained with a camera based on the contents of the label 600 will be described with reference to FIG. 12 and FIG. 13. The above-described various application examples including the application example regarding the wagon and the label and an application example regarding a plurality of articles are applicable.

Initially, an example of the overall configuration of the conveyance system will be described with reference to FIG. 12. FIG. 12 is a schematic diagram that shows an example of the overall configuration of the conveyance system including the transfer robot 100.

As shown in FIG. 12, the conveyance system 1 includes the transfer robot 100, a host management apparatus 2, a network 3, a communication unit 4, and an environmental camera 5. The conveyance system 1 is a system that conveys an article to be conveyed with the transfer robot 100. The conveyance system 1 includes a control system in the present configuration example. In the case of this example, the control system may, for example, correspond to the transfer robot 100 and the host management apparatus 2 or correspond to component elements of a control system provided in the transfer robot 100 and the host management apparatus 2.

The transfer robot 100 is connected to the host management apparatus 2 via the communication unit 4 and the network 3. The network 3 is a wired or wireless local area network (LAN) or a wide area network (WAN). The host management apparatus 2 and the environmental camera 5 are connected to the network 3 in a wired or wireless manner. As is apparent from such a configuration, each of the transfer robot 100, the host management apparatus 2, and the environmental camera 5 includes a communication unit. The communication unit 4 is, for example, a wireless LAN unit installed in each environment. The communication unit 4 may be, for example, a general communication device, such as a WiFi (registered trademark) router.

The host management apparatus 2 is capable of connecting with the transfer robot 100 by wireless communication. The host management apparatus 2 is a management system that manages a plurality of the transfer robots 100. The host management apparatus 2 may include a controller 2a that executes such control. The controller 2a may be implemented by, for example, an integrated circuit and may be, for example, implemented by a processor, such as an MPU and a CPU, a working memory, a nonvolatile storage device, and the like. When a program for control to be executed on the processor is stored in advance in the storage device and the processor loads the program onto the working memory and runs the program, it is possible to achieve the function of the controller 2a. The controller 2a may be referred to as a control computer.

The conveyance system 1 is capable of efficiently controlling the transfer robots 100 while causing the transfer robots 100 to autonomously move in the autonomous moving mode in a predetermined facility or causing the transfer robots 100 to move based on user operation in the user operation mode. The facility can mean facilities of various types. Examples of the facility include medical and welfare facilities, such as a hospital, a rehabilitation center, a nursing-care facility, and an elderly living facility, commercial facilities, such as a hotel, a restaurant, an office building, an event site, and a shopping mall, and other complex facilities.

To execute such efficient control, a plurality of the environmental cameras 5 may be installed in a facility. The environmental camera 5 acquires an image of a range in which a human or the transfer robot 100 moves and outputs image data describing the image. The image data may be still image data or moving image data. In the case of still image data, still image data is obtained at image capture intervals. In the conveyance system 1, the host management apparatus 2 collects images acquired by the environmental cameras 5 and information based on the images. Images used to control the transfer robot 100 may be images and the like acquired by the environmental cameras 5 and directly sent to the transfer robot 100. The environmental cameras 5 can be provided at passages and entrances in the facility as surveillance cameras.

In the conveyance system 1, images acquired by the environmental cameras 5 can be used to monitor a human and the transfer robot 100 and can also be used to read the QR code 606 on the label 600.

The environmental cameras 5 included in the conveyance system 1 sometimes capture an image of a state including the label 600 attached to the label attachment portion 503 of the wagon 500. Alternatively, when one or multiple places for loading the wagon 500 are set in advance in a facility as an operation rule and the environmental camera 5 is installed at each of the one or multiple locations in advance, it is easy to obtain such an image of a state including the label 600. Particularly, as an operation rule, to make it easy for the environmental cameras 5 at the one or multiple locations to capture an image of a state including the label 600, not only a loading place but also an orientation in which the wagon 500 is placed may be determined in advance to a direction that falls within the angle of view of each environmental camera 5.

When the environmental camera 5 captures an image of a state including the label 600, actually, an image of a state including at least the QR code 606, the environmental camera 5 sends image data of the image to the host management apparatus 2 as in the case of the other images. The host management apparatus 2 receives the image data, and the controller 2a performs image analysis to detect conveyance destination information from the QR code 606. When the environmental camera 5 is capable of performing image analysis, the host management apparatus 2 receives, from the environmental camera 5, conveyance destination information obtained through the image analysis by the environmental camera 5.

Next, an example of control over the transfer robot 100, which is executed by the host management apparatus 2, will be described with reference to FIG. 13. FIG. 13 is a flowchart for illustrating an example of control over the transfer robot 100, which is executed by the host management apparatus 2 in the conveyance system 1 of FIG. 12. In the example of this control as well, various application examples as described with reference to FIG. 8 are applicable.

Initially, the user attaches the label 600 at the label attachment portion 503 and moves the wagon 500 to a predetermined place or the user goes to a place where the wagon 500 is placed and attaches the label 600 at the label attachment portion 503. As a result, the user is able to issue conveying instructions to the transfer robot 100 that reads the label 600. After that, as will be described below, the conveyance system 1 performs conveyance of the wagon 500.

The environmental camera 5 initially captures an image of a state including the label 600, actually, an image of a state including at least the QR code 606, and sends image data of the image to the host management apparatus 2. The host management apparatus 2 receives the image data (step S21). The controller 2a of the host management apparatus 2 performs image analysis on the received image data and detects conveyance destination information indicated by the QR code 606 (step S22). Thus, the controller 2a recognizes a destination of conveyance that is, a destination.

Subsequently, the controller 2a chooses the transfer robot 100 that performs conveyance (step S23) and sets a route to convey the wagon 500 from the location of the wagon 500 to the destination of conveyance indicated by the conveyance destination information (step S24). In step S23, the controller 2a may check the location of each transfer robot 100 in operation and, for example, choose the transfer robot 100 closest to the current location of the wagon 500 for the transfer robot that performs conveyance; however, a method of choosing the transfer robot that performs conveyance is not limited. For example, the controller 2a may choose the transfer robot in consideration of a conveying schedule of each transfer robot 100.

The order of step S23 and step S24 is not limited. However, when a route is chosen and then the transfer robot is chosen, it is possible to, for example, select the transfer robot that can complete conveyance the earliest. On the other hand, when a transfer robot with no conveying schedule is selected as the transfer robot that performs conveyance, it is possible to complete conveyance the earliest even when a route is set thereafter. The description is made on the assumption that the performance of each of the transfer robots 100 in operation is the same. Transfer robots with different performance of maximum speed, running stability, or the like may be operated in the same facility. In this case, the transfer robot that performs conveyance may be chosen additionally in consideration of the performance of each transfer robot.

Subsequently, the controller 2a, for example, sends the set route to the transfer robot 100 that performs conveyance and controls the transfer robot 100 such that the transfer robot 100 autonomously moves along the set route (step S25), and the process ends. In step S25, the controller 2a controls the transfer robot 100 such that the transfer robot 100 conveys the wagon 500 to the destination of conveyance as described in step S14 to step S15 in FIG. 8.

The controller 2a is capable of controlling the transfer robot 100 by, for example, providing instructions for a series of operations to the transfer robot 100. The series of operations includes autonomous movement to the location of the wagon 500, loading of the wagon 500, autonomous movement to the destination of conveyance, and unloading of the wagon 500. At this time, the controller 2a may provide instructions to the transfer robot 100 based on image data captured each time with the environmental camera 5. For example, the controller 2a designates a route only, and subsequent autonomous movement is left for the transfer robot 100. Thus, the controller 2a may execute only part of control. For example, the control computer 101 may receive a set route from the host management apparatus 2 and set the route. Then, the control computer 101 may control the drive unit such that the transfer robot 100 autonomously moves, and, when the transfer robot 100 reaches the location of the wagon 500, control the lifting and lowering mechanism 140 such that the lifting and lowering mechanism 140 loads the wagon 500. After that, the control computer 101 may control the drive unit such that the wagon 500 is conveyed along the set route, and control the lifting and lowering mechanism 140 at the destination of conveyance to unload the wagon 500.

A conveyance system may be configured such that the conveyance system 1 does not include the host management apparatus 2. In this case, the conveyance system may include at least the transfer robot 100 and the environmental camera 5 capable of performing wireless communication with the transfer robot 100. In this configuration example as well, similarly, the environmental camera 5 is capable of obtaining conveyance destination information or capable of obtaining data that is a source of conveyance destination information, and the transfer robot 100 may acquire, from the environmental camera 5 through wireless communication, the conveyance destination information obtained in this way or the data that is a source of conveyance destination information. The control computer 101 of the transfer robot 100 may control the drive unit such that the transfer robot 100 autonomously moves to convey the wagon 500 to the destination of conveyance indicated by the conveyance destination information based on the acquired conveyance destination information or data that is a source of conveyance destination information.

Regardless of whether the conveyance system includes the host management apparatus 2, the conveyance system may include a sensor of another type, which detects conveyance destination information, instead of the environmental camera 5. In these configuration examples as well, similarly, the sensor of another type is capable of obtaining conveyance destination information or obtaining data that is a source of conveyance destination information. Thus, the transfer robot 100 or the host management apparatus 2 can acquire, from the sensor of another type, conveyance destination information obtained in this way or data that is a source of conveyance destination information. The transfer robot 100 can convey the wagon 500 to a destination of conveyance indicated by the conveyance destination information or conveyance destination information detected from the data that is a source of conveyance destination information.

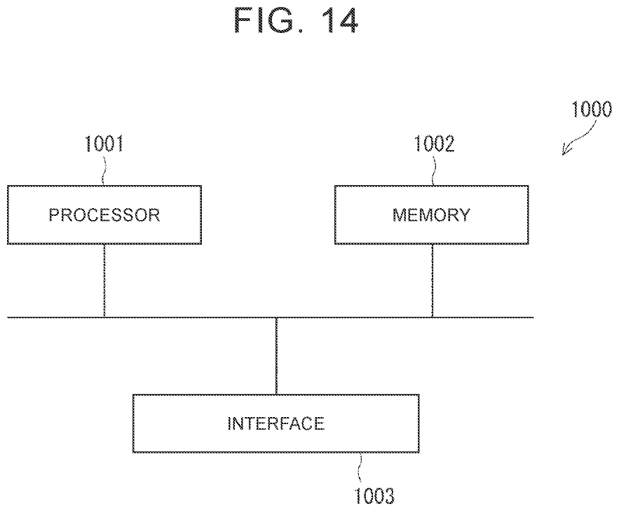

Each of the control computer 101 of the transfer robot 100, the control computer 101 of the transfer robot 100a, the host management apparatus 2, and the sensors such as the environmental camera 5 according to the above-described embodiment may include, for example, the following hardware configuration. FIG. 14 is a diagram that shows an example of the hardware configuration of an apparatus.

The apparatus 1000 shown in FIG. 14 may include a processor 1001, a memory 1002, and an interface 1003. The interface 1003 may include, for example, a communication interface and interfaces with the drive unit, the sensors, the input/output device, and the like, needed according to the apparatus.

The processor 1001 may be, for example, an MPU, a CPU, a graphics processing unit (GPU), or the like. The processor 1001 may include a plurality of processors. The memory 1002 is made up of, for example, a combination of a volatile memory and a nonvolatile memory. Functions in each apparatus may be implemented by the processor 1001 loading a program stored in the memory 1002 and running the program while exchanging necessary information via the interface 1003.

The above program includes a command set (or software code) for causing a computer to execute one or more functions described in the embodiment when the program is loaded onto the computer. The program may be stored in a non-transitory computer-readable medium or tangible storage medium. Nonrestrictive examples of the computer-readable medium or tangible storage medium include memory technologies, such as a random-access memory (RAM), a read-only memory (ROM), a flash memory, a solid-state drive (SSD), and others, optical disk storages, such as a CD-ROM, a digital versatile disc (DVD), a Blu-ray (registered trademark) disc, and others, and magnetic storage devices, such as a magnetic cassette, a magnetic tape, a magnetic disk storage, and others. The program may be transmitted on a transitory computer-readable medium or communication medium. Nonrestrictive examples of the transitory computer-readable medium or communication medium include electrical, optical, acoustic, and other-type propagation signals.

The disclosure is not limited to the above-described embodiment and may be modified as needed without departing from the scope of the disclosure.

What is claimed is:

1. A control system that controls a conveyance system including a transfer robot capable of autonomously moving and capable of conveying an article to be conveyed, the control system comprising:
an acquisition unit configured to acquire conveyance destination information from a wagon capable of accommodating the article, the conveyance destination information indicating a destination of conveyance of the article; and
a controller configured to cause the transfer robot to autonomously move such that the transfer robot conveys the wagon to the destination of conveyance indicated by the conveyance destination information.

2. The control system according to claim 1, wherein:
the wagon includes a label on which the conveyance destination information is printed; and
the acquisition unit includes a first sensor configured to detect the conveyance destination information from the label or a communication unit configured to receive the conveyance destination information detected by a second sensor.

3. The control system according to claim 1, wherein:
the wagon includes a label on which the conveyance destination information is printed; and
the acquisition unit includes a first camera configured to obtain image data by capturing an image on the label or a communication unit configured to receive image data obtained by capturing an image on the label with a second camera; and the controller is configured to detect the conveyance destination information from the image data.

4. The control system according to claim 2, wherein the conveyance destination information is printed on a first side of the label, and moving destination information indicating a destination of movement to which the wagon is moved after being conveyed to the destination of conveyance indicated by the conveyance destination information is printed on a second side of the label.

5. The control system according to claim 2, wherein a two-dimensional code or a one-dimensional code, indicating the destination of conveyance, is printed on the label.

6. The control system according to claim 2, wherein:
the label has a tab; and
at least one of a location of the tab provided, a color of the tab, and a shape of the tab is varied for each destination of conveyance.

7. The control system according to claim 2, wherein the label is a label on which a character indicating the destination of conveyance is printed or a label in a color indicating the destination of conveyance.

8. The control system according to claim 2, wherein the wagon includes a label attachment portion that allows the label to be attached in a replaceable state.

9. The control system according to claim 1, wherein:
the wagon includes a display device showing the conveyance destination information; and
the acquisition unit includes a first sensor configured to detect the conveyance destination information from the display device or a communication unit configured to receive the conveyance destination information detected from the display device by a second sensor.

10. The control system according to claim 1, wherein:
the wagon includes a display device showing the conveyance destination information;
the acquisition unit includes a first camera configured to obtain image data by capturing an image on the display device or a communication unit configured to receive image data obtained by capturing an image on the display device with a second camera; and
the controller is configured to detect the conveyance destination information from the image data.

11. The control system according to claim 1, wherein:
the wagon includes a radio frequency identification (RFID) tag on which the conveyance destination information is recorded; and
the acquisition unit includes a first RFID reader configured to read the conveyance destination information from the RFID tag or a communication unit configured to receive the conveyance destination information read from the RFID tag with a second RFID reader.

12. The control system according to claim 1, wherein:
the conveyance destination information includes information indicating a plurality of destinations of conveyance; and
the controller is configured to cause the transfer robot to autonomously move such that the transfer robot conveys the wagon along a route including the plurality of destinations of conveyance indicated by the conveyance destination information.

13. The control system according to claim 1, wherein:
the wagon includes a storage portion that accommodates the article and a supporting portion that supports the storage portion in a state where a space for allowing at least part of a chassis of the transfer robot to enter is provided under the storage portion; and
the transfer robot includes a lifting and lowering mechanism that loads and unloads the wagon to and from a top surface side of the at least part of the chassis.

14. A control method comprising:
acquiring, by a control system that controls a conveyance system including a transfer robot capable of autonomously moving and capable of conveying an article to be conveyed, conveyance destination information indicating a destination of conveyance of the article from a wagon capable of accommodating the article; and
causing the transfer robot, by the control system, to autonomously move such that the transfer robot conveys the wagon to the destination of conveyance indicated by the conveyance destination information.

15. The control method according to claim 14, wherein:
the wagon includes a label on which the conveyance destination information is printed; and
the control method further comprises detecting, by a first sensor, the conveyance destination information from the label or receiving, by a communication unit, the conveyance destination information detected from the label by a second sensor.

16. The control method according to claim 14, wherein:
the wagon includes a label on which the conveyance destination information is printed; and
the control method further comprises:
obtaining, by a first camera, image data by capturing an image on the label or receiving, by a communication unit, image data obtained by capturing an image on the label with a second camera; and
detecting the conveyance destination information from the image data.

17. The control method according to claim 14, wherein:
the wagon includes a display device showing the conveyance destination information; and
the control method further comprises detecting, by a first sensor, the conveyance destination information from the display device or receiving, by a communication unit, the conveyance destination information detected by a second sensor.

18. The control method according to claim 14, wherein:
the wagon includes a display device showing the conveyance destination information; and
the control method further comprises:
obtaining, by a first camera, image data by capturing an image on the display device or receiving, by a communication unit, image data obtained by capturing an image on the display device with a second camera; and
detecting the conveyance destination information from the image data.

19. The control method according to claim 14, wherein:
the wagon includes a radio frequency identification (RFID) tag on which the conveyance destination information is recorded; and
the control method further comprises reading, by a first RFID reader, the conveyance destination information from the RFID tag or receiving, by a communication unit, the conveyance destination information read from the RFID tag with a second RFID reader.

20. A non-transitory storage medium storing instructions executable on one or more processors of a computer of a control system that controls a conveyance system including a transfer robot capable of autonomously moving and capable of conveying an article to be conveyed, the instructions causing the one or more processors to execute functions comprising:
(i) inputting conveyance destination information indicating a destination of conveyance of the article and acquired from a wagon capable of accommodating the article; and
(ii) causing the transfer robot to autonomously move such that the transfer robot conveys the wagon to the destination of conveyance indicated by the input conveyance destination information.
